(19) United States
(12) Patent Application Publication
(10) Pub. No.: US 2006/0058580 A1
(43) Pub. Date: Mar. 16, 2006

(54) Resectoscope electrode that is longitudinally displaced by a carriage

(75) Inventors: Jens Reichenbach, Hamburg (DE); Dido Arnim Zweibruck, Hamburg (DE); Manfred Held, Hamburg (DE)

Correspondence Address: RANKIN, HILL, PORTER & CLARK LLP, 4080 ERIE STREET, WILLOUGHBY, OH, 44094-7836, US

(73) Assignee: OLYMPUS WINTER & IBE GMBH, Hamburg (DE)

(21) Appl. No.: 11/222,325

(22) Filed: Sep. 8, 2005

(30) Foreign Application Priority Data
Sep. 16, 2004 (DE) 102004045337.3-35

(51) Int. Cl. A61B 1/00

(52) U.S. Cl. 600/104

(57) ABSTRACT

A resectoscope having an electrode longitudinally displaceable in a stem. The electrode is electrically contacted at its proximal end 31, in a carriage that is fitted with a down-pointing finger grip, by a contacting system. The resectoscope further includes an hf-power cable 15 affixed to and suspended from the carriage. The contacting system is a separate reversal device affixable in the distal direction selectively by either of its two ends to the carriage. The particular distal end being fitted with a recess to receive the electrode and a cable post 13 is situated nearer one of the ends.

### BACKGROUND OF THE INVENTION

1. Field of the Invention

The present invention relates to resectoscopes and, more particularly, to resectoscopes having longitudinally displaced electrodes.

2. Description of Related Art

Resectoscopes having longitudinally displaceable electrodes are used in urology, in particular to remove the prostate. The electrode is typically designed with a high-frequency operated cutting loop mounted at its distal end. This electrode is longitudinally adjustable, for which purpose use is made of a finger grip driven carriage.

Such resectoscopes are widely used and come in two designs, namely the “active” resectoscopes prevalent in Europe and the “passive” ones prevalent in the USA. Actuation is implemented by the fingers of one hand being applied to the finger grip mounted on the carriage and also to a further finger grip which, as regards the active design, is affixed to the resectoscope proximally from the carriage, while being distally from the carriage in the passive design. It follows that as regards the active design, the finger grip should be affixed to the carriage as distally as possible while in the passive design it should be as proximally as possible.

In resectoscopes of these species, the cable feeding the hf power is suspended from the carriage, thereby attaining a number of advantages, though also entailing the drawback that the cable is disadvantageously close to the finger grip.

Accordingly, the cable affixation to the carriage should be as proximal as possible in the active design and as distal as possible in the passive design. As a result, different carriages are required for the active and passive designs, leading to higher costs in parts.

### SUMMARY OF THE INVENTION

The present invention is directed toward a resectoscope of the above species that allows substantial savings in costs for both designs.

In accordance with the present invention, the electrical connection system between the cable post and the electrode is a reversal device which can be mounted selectively in two directions on the carriage, the cable post being situated closer to one end of the carriage, and therefore, according to the insert position of the reversal device, farther distally or farther proximally. Depending on the position of the inserted reversal device, the cable post will be farther distally or farther proximally. By assembling the reversal device to the carriage in either inserted position, the carriage may be matched in very economical manner to the resectoscope's passive or active design.

The reversal device may be affixed in widely arbitrary manner to the carriage, for instance being screwed-connected to it or the like. The reversal device may be plugged into an elongated slot, the two plug-in directions being determined by the slot's lengthwise extension.

The hf power cable must be connected at one end to an hf generator and is firmly affixed at the other end to the reversal device. Advantageously, however, the reversal device is fitted with an electrical jack receiving the cable plug.

Depending on the reversal device's direction of insertion, it makes electrical contact at either end with the distally incoming electrode. The reversal device may be fitted at both ends with recesses receiving the electrode and making contact with it. The recess making electrical contact with the electrode may be geometrically reversible such that, depending on the reversal device's direction of insertion, it shall be affixable to that end which points distally.

### BRIEF DESCRIPTION OF THE DRAWINGS

These and further features of the invention will be apparent with reference to the following description and drawings, wherein:

FIG. 1 is a sideview of a resectoscope of the invention with a carriage exhibiting the active design,

FIG. 2 is a sideview of a resectoscope of the invention with carriage exhibiting the passive design,

FIG. 3 is an enlarged axial view of the carriage of FIG. 1 or 2 exhibiting the inserted reversal device, and

FIG. 4 is a bottom view of the reversal device of FIG. 3.

### DETAILED DESCRIPTION OF THE PREFERRED EMBODIMENTS

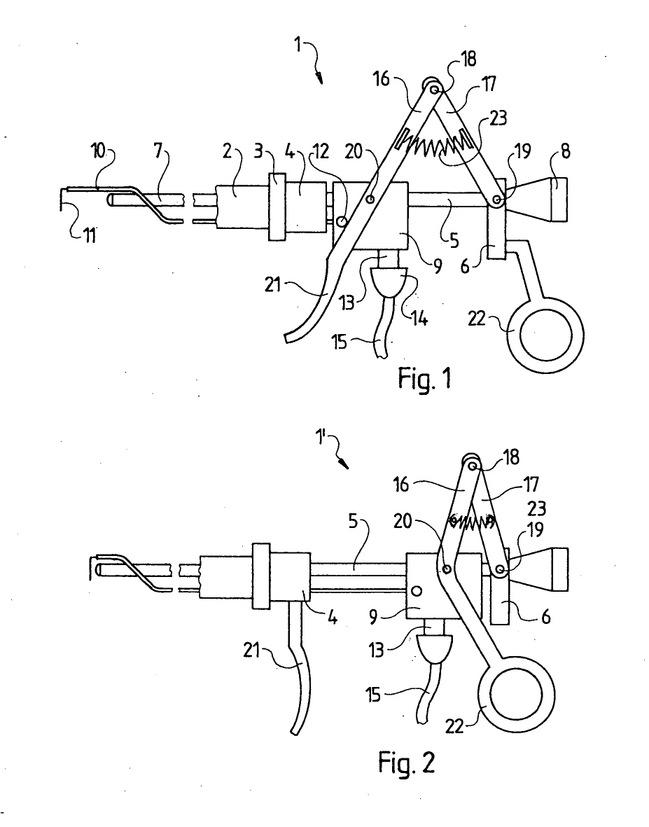

FIG. 1 is a substantially schematic view of an urological resectoscope 1 having a tubular stem 2 that is affixable by a connection element 3 to a main stub 4. For the sake of drawing simplicity, further stem details such as an additional inner stem conveying liquids and rinsing-liquid intakes and outlets have been omitted.

The main stub 4 is connected by a guide tube 5 to an end plate 6 proximally spaced apart from it. An optics 7 comprising a proximal ocular lens 8 runs through the guide tube 5 and the stem 2.

A carriage 9 rests on the guide tube 5 and is longitudinally displaceable between the main stub 4 and the end plate 6. An electrode 10 rests in a longitudinally displaceable manner in the stem 2 and is fitted at its distal end with a cutting loop 11. The proximal electrode end runs through the main stub 4 and is inserted, parallel to the guide tube 5, into the carriage 9 in which it is detachably affixed by a clamping screw 12. Inside the carriage 9, the electrode makes electrical contact with a cable post 13 to which a cable 15 is affixed by a cable plug 14, the cable at its other end being connected to an omitted high frequency generator.

The shown resectoscope exhibits the active design. Therein the carriage 9 is pulled proximally by a finger against a spring force and, once released, will automatically move distally due to the spring force.

For that purpose, a long lever 16 and a short lever 17 are used. The levers hinge on each other at 18 and rest at 19 against the end plate 6 and at 20 against the carriage 9. At its end projecting underneath the pivot 20, the long lever 16 subtends a finger grip 21 near the carriage 9. A thumb ring 22 is mounted to the end plate 6. The levers 16 and 17 are biased apart by a compression spring 23. When the surgeon seizes by thumb and index finger, respectively, the thumb ring 22 and the finger grip 21, then the carriage 9 may be displaced in the proximal direction by compressing the ring and grip against the force of the spring 23. When the compression is lifted, the carriage 9 automatically will move in the distal direction. Electrode 10 and cutting loop 11 then are moved together with the carriage.

FIG. 2 shows a resectoscope 1′ which contains several slight modifications of that of FIG. 1. To the extent possible the same references are used also in FIG. 2. The resectoscope 1′ exhibits the passive design. The required changes are discussed immediately below.

The finger grip is 21 affixed to the main stub 4. The long lever 16 subtends at its lower end a proximal angle with the vertical and is fitted at the end with the thumb ring 22.

As regards the resectoscope 1′, compressing the fingers displaces the carriage in the distal direction, while releasing the finger compression moves the carriage in the proximal direction.

Comparison of FIGS. 1 and 2 indicates that in the design of FIG. 1, the grip element mounted on the carriage 9, that is the finger grip 21, is situated distally from the cable post 13. As regards the design shown in FIG. 2, the thumb ring 22 mounted on the carriage 9 is situated proximally from the cable post 13. Both designs provide that the carriage motion in the proximal direction (FIG. 1) respectively in the distal direction (FIG. 2) do entail pivoting the long lever 16 in a manner that the finger grip 21 (FIG. 1) respectively the thumb ring 22 (FIG. 2) will in each case approach the cable 15.

Accordingly, to prevent hampering physical contact with the cable 15, the cable post 13 advantageously shall be as proximally away from the carriage 9 in the design of FIG. 1 as shown in this Figure. As regards the design of FIG. 2, the cable post, as shown therein, shall be mounted as distally as possible on the carriage 9.

In this way a different configuration of the carriage 9, with a proximal/distal cable post 13 for the two designs of FIGS. 1 and 2 has been attained.

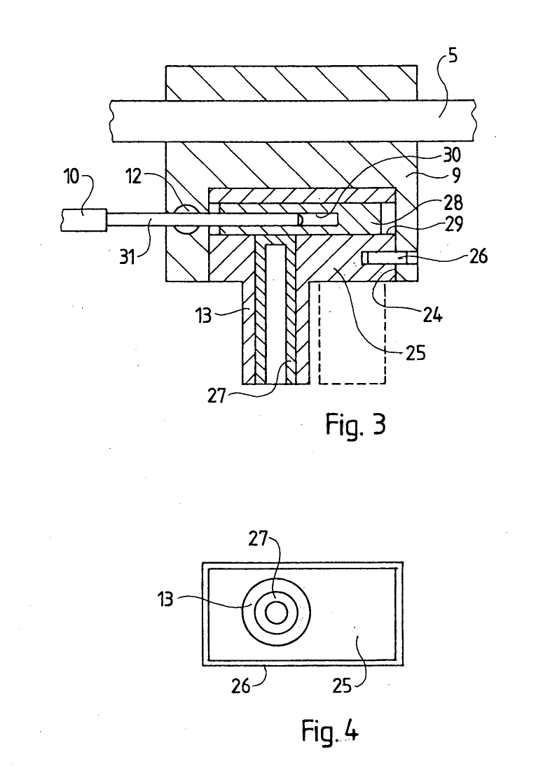

FIG. 3 shows a design of the carriage 9 that is applicable both to the resectoscope configuration of FIG. 1 and of FIG. 2.

As shown by FIG. 3, the carriage 9, which typically is made of a solid, electrically insulating material, comprises a rectangular elongated slot 24 milled out from below which receives a rectangular reversal device or element 25 (FIG. 4) that is secured in place by a securing pin 26.

As for the carriage 9, the reversal device 25 also is made of an electrically insulating material and it is fitted in the zone of the cable post 13 situated at the reversal device 25 with a metallic electrical jack 27 that shall receive a contact pin of the cable plug 14. Inversely, instead of the jack 27, a contact pin may be used that shall engage a matching jack at the connector element 14. The electrical jack 27 makes contact with an electrical jack 28 configured perpendicularly to the cable post 13 in a borehole 29 in the reversal device 25. The jack 28 is fitted with distally open borehole 30 receiving and making electrical contact with the proximal end 31 of the electrode 10.

In the design of FIG. 3, the reversal device 25 is inserted in such manner into the carriage 9 that the cable post 13 is situated on the left, that is nearer the distal end of the carriage 9. In this configuration the carriage 9 is appropriate for the passive resectoscope design of FIG. 2.

The reversal device 25 may be removed from the carriage 9 in order to be rotated by 180° whereby, as shown in dashed lines in FIG. 3, the cable post shall be situated farther proximally. Thereupon, the carriage 9 will be appropriate for the resectoscope design of FIG. 1. In the process the electrode jack 28 is removed from the borehole 29 of the reversal device 25 and rotated by 180° whereby the borehole 30 once more is open in the distal direction.

The carriage embodiment shown in FIGS. 3 and 4 may be modified. The reversal device 25 shown in the form of connection system also may be affixed in another way to the carriage 9, for instance it might constitute a separate lower half of this carriage, and be screwed to the upper half. The elongated slot 24 might be oval instead of rectangular, as shown. Instead of the illustrated embodiment mode, the electrode jack 28 may be insertable in two directions, also it may be firmly affixed in the reversing component 25 and be fitted, for instance with a continuous borehole 30, at both of its ends, to receive the electrode end 31. Other snap-in elements or also screw elements may be used instead of the securing pin 26 to secure the reversal component 25 to the carriage 9.

1. A resectoscope having an electrode longitudinally displaceable in a stem, a proximal end of said electrode being electrically connected to an hf-power cable, in a carriage that is fitted with a down-pointing finger grip, by a contacting system, wherein the hf-power cable is affixed to and suspended from said carriage, wherein the contacting system is a separate reversal device affixable to the carriage, said reversal device including first and second ends and is disposed in said carriage such that the first end points in a distal direction while the second end includes a recess to receive the electrodes, and wherein a cable post 13 is disposed nearer one of said first and second ends that an other of said first and second ends.

2. The resectoscope as claimed in claim 1, wherein the reversal device is inserted from below into a slot that is elongated in a thrust direction within the carriage.

3. The resectoscope as claimed in claim 1, wherein the reversal device is secured in the elongated slot by a snap-in element.

4. The resectoscope as claimed in claim 1, wherein the cable post is an electrical jack receiving a cable plug.

5. The resectoscope as claimed in claim 1, wherein the recess for the electrode in the reversal device is reversibly affixed at either end.
